(19) United States
(12) Plant Patent Application Publication
(10) Pub. No.: US 2006/0230483 P1
(43) Pub. Date: Oct. 12, 2006

(54) Ananas Lucidus Miller plant named 'Xanda'

(50) ANANAS LUCIDUS; ANANAS

(76) Inventor: Estevam Emygdio de Castro Filho, Fortaleza (BR)

Correspondence Address: Paul S. Rooy, 2620 S. Peninsula Dr., Daytona Beach, FL, 32118, US

(21) Appl. No.: 11/102,919

(22) Filed: Apr. 11, 2005

(51) Int. Cl. A01H 5/00

(52) U.S. Cl. PLT/370

(57) ABSTRACT

The Ananas Lucidus Miller ‘Xanda’ exhibits lance-shaped leaves and straight pipes (Canaliformes) wrapped around the stalk, thornless, with the exception of the terminal thorn. The outer skin of the superior leaves presents a uniform waxy layer, smooth and has a reddish color in the central part of the leaf and dark reddish at the edges. The inferior outer skin is composed of grooves in the longitude direction, where there can be found the stomas. It is covered by tricomas peltadosescalformes, composed by a white-silverish and continuous fuzz. The peduncle is the part of the plant which develops like an extension of the stalk, making a connection between the stalk and the fruit. The peduncle sustains the inflorescence, and later on the fruit. The fruit is green colored at the base and red in the middle and the top. With the presence of tricomas a great quantity of offspring are presented.

### LATIN NAME OF THE GENUS AND SPECIES OF THE PLANT CLAIMED

The plant genus is Ananas Miller. The plant species is Lucidus.

### FIELD OF THE INVENTION

The present invention comprises a new and distinct variety of Ananas Lucidus Miller which is named ‘Xanda’. Its genus is Ananas Miller. Its market class is that of ornamental potted, landscape, foliage and flower cuttings plants.

### DESCRIPTION OF PRIOR ART

The mother plants that the cultivated ‘Xanda’ is related to are of the species Ananas Lucidus Miller.

The following characteristics were obtained by observing the prior art Lucidus and instant Xanda plants, described below according to it's progress:
A—The plant (vegetative progress stage):
1) Leaf position:
   Lucidus.—Erect.
   Xanda.—Erect.
2) Height of the plant:
   Lucidus.—92 cm.
   Xanda.—92 cm.
3) Plant diameter:
   Lucidus.—74 cm.
   Xanda.—74 cm.
B—The leaf (vegetative progress stage):
1) Leaf length:
   Lucidus.—82 cm.
   Xanda.—81.5 cm.
2) Leaf width (in the middle of the leaf):
   Lucidus.—36 mm.
   Xanda.—38 mm.
3) Presence of thorns on the leaves:
   Lucidus.—Only the terminal.
   Xanda.—Only the terminal.
C—Inflorescence (stage of development with flowers in the middle of the inflorescence):
1) Length of the inflorescence:
   Lucidus.—40 mm.
   Xanda.—40 mm.
2) Diameter of the inflorescence (middle part):
   Lucidus.—28.5 mm.
   Xanda.—28 mm.
3) Length of the peduncle:
   Lucidus.—60 mm.
   Xanda.—55 mm.
4) Diameter of the peduncle (middle part):
   Lucidus.—10 mm.
   Xanda.—11 mm.
5) Length of the crown:
   Lucidus.—20 mm.
   Xanda.—20 mm.
6) Number of bracts on the peduncle:
   Lucidus.—9.
   Xanda.—10.
7) Number of bracts at the base of the fruit:
   Lucidus.—3.
   Xanda.—3.
8) Number of berries on the fruit:
   Lucidus.—69.
   Xanda.—80.
D—Presence of offspring (mature fruit stage):
1—Number of Offspring:
   Lucidus.—High Quantity (more that 2).
   Xanda.—High Quantity (more than 2).
2—Number of Sapling-Offspring:
   Lucidus.—High Quantity (more than 2).
   Xanda.—High Quantity (more than 2).
3—Number of saplings at the base of the fruit:
   Lucidus.—Medium Quantity (5 to 10).
   Xanda.—Medium Quantity (5 to 10).
Uniformity: Ananas Lucidus Miller ‘Xanda’ was first reproduced in 1997. The plant exhibits a semiperene cycle. The plant has proven stable in reproduction. Eight generations of Xanda have been grown since 1997, and the propagules are identical to the original ‘Xanda’.

### SUMMARY OF THE INVENTION

The major characteristics of ‘Xanda’ include erectness as a plant, fruit less than 15 cm in height when at the adult stage, straight, dark reddish thornless leaves terminating in thorns, and a long, dark reddish stalk.

The following traits which have been repeatedly observed are determined to be basic characteristics of ‘Xanda’, which in combination distinguish ‘Xanda’ from other Ananas Lucidus Miller of the same general type.
1. Leaf upper surfaces are brilliant red-orange in the middle, ranging from red RHS 48B at the base through red RHS 43A at mid leaf to red RHS 46A close to the tip, with dark brown RHS 200A borders, lightening towards the tip.
2. The ‘Xanda’ stalk is long and colored green-yellow RHS 1D.
3. The peduncle starts with green-yellow RHS 1D color, from the middle onwards turns into orange-red RHS 35B, and ends with Red RHS 39A.

### BRIEF DESCRIPTION OF THE DRAWINGS

The invention, together with the other objects, features, aspects and advantages thereof will be more clearly understood from the following in conjunction with the accompanying drawings.

Four sheets of drawings are provided. Sheet one contains FIG. 1. Sheet two contains FIG. 2. Sheet three contains FIGS. 3 and 4. Sheet 4 contains FIGS. 5 and 6.

FIG. 1 is a color photograph of a front view of an Ananas Lucidus Miller ‘Xanda’ plant which has not yet flowered.

FIG. 2 is a color photograph of a top view of an Ananas Lucidus Miller ‘Xanda’ plant which has flowered.

FIG. 3 is a color photograph of an Ananas Lucidus Miller ‘Xanda’ plant which has flowered on the left, and an Ananas Lucidus Miller ‘Xanda’ plant which has not yet flowered on the right.

FIG. 4 is a color photograph close-up view of an Ananas Lucidus Miller ‘Xanda” fruit.

FIG. 5 is a color photograph of an Ananas Lucidus Miller ‘Xanda” showing the peduncle coloration and roots.

FIG. 6 is a color photograph of an Ananas Lucidus Miller ‘Xanda” showing the roots.

### BOTANICAL DESCRIPTION OF THE PLANT

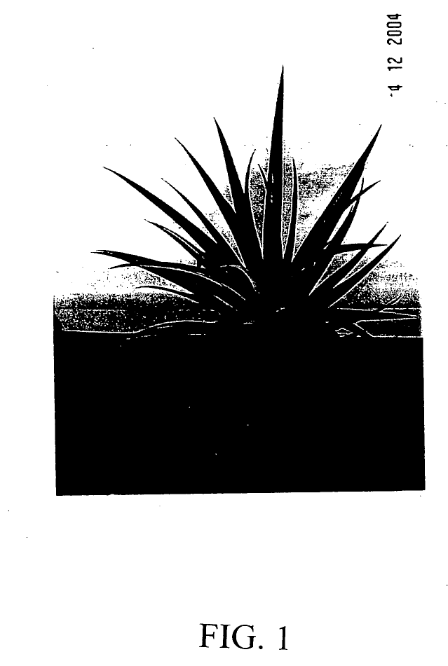

FIG. 1 is a color photograph of a front view of an Ananas Lucidus Miller ‘Xanda’ plant which has not yet flowered. The plant is ten months old. The distinctive leaf color is illustrated in FIG. 1. Leaf upper surfaces are brilliant red-orange in the middle, ranging from red RHS 48B at the base through red RHS 43A at mid leaf to red RHS 46A close to the tip, with dark brown RHS 200A borders, lightening towards the tip. This brilliant red-orange coloration is in stark contrast to prior art Ananas Lucidus Miller plants, which exhibit a dull rust-brown center leaf coloration. Leaf lower surfaces are grayed-green RHS 197A, with dull red stripes RHS 48C at the base, RHS 48A at midleaf, and 52B toward the tip.

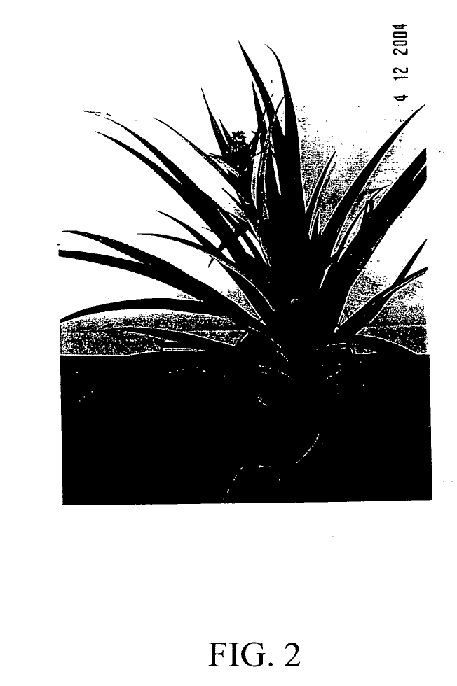

FIG. 2 is a color photograph of a top view of an Ananas Lucidus Miller ‘Xanda’ plant which has flowered. The plant is ten months old, and was gassed with etephon four months previously. One method to induce flowering in Ananas Lucidus Miller ‘Xanda’ plants is to gas with etephon. Upon flowering, the brilliant red-orange coloration of the leaves and peduncle increases in intensity, and darkens slightly. As the Ananas Lucidus Miller ‘Xanda’ plants age, their coloration darkens.

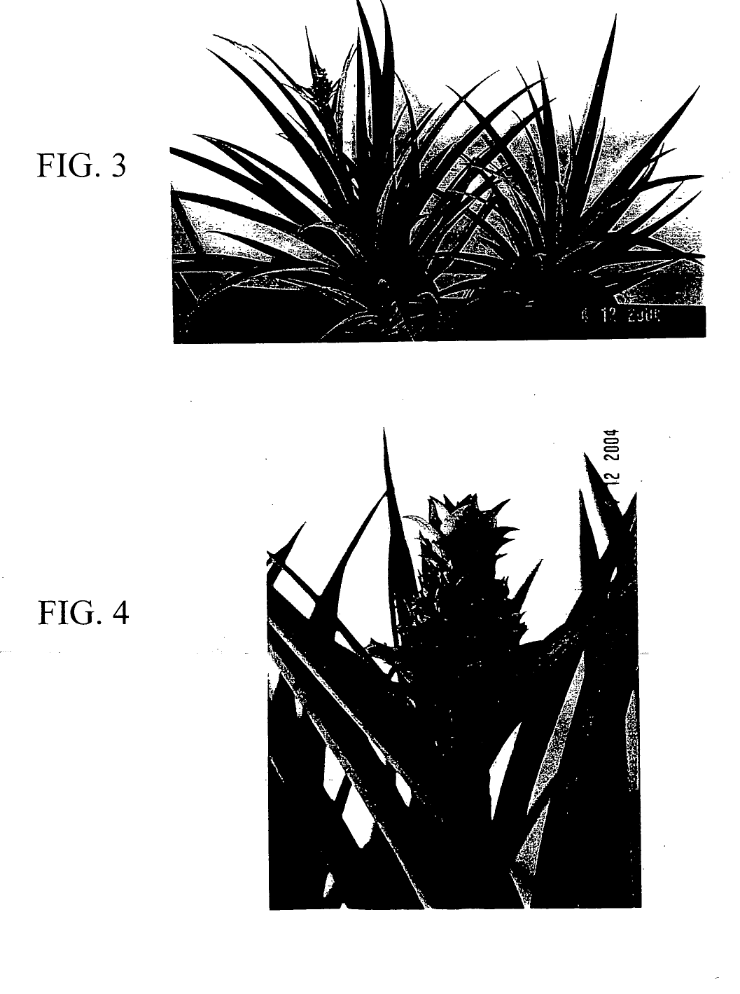

FIG. 3 is a color photograph of an Ananas Lucidus Miller ‘Xanda’ plant which has flowered on the left, and an Ananas Lucidus Miller ‘Xanda’ plant which has not yet flowered on the right. The flowering plant is ten months old; the non-flowering plant is seven months old.

FIG. 4 is a color photograph close-up view of an Ananas Lucidus Miller ‘Xanda” fruit, which is colored red RHS 48C- 49. This specimen came from a ‘Xanda’ plant ten months old. One of the characteristics of the Ananas Lucidus Miller ‘Xanda” is the height of the fruit, which does not exceed 15 cm. The leaflets stemming off the peduncle exhibit a central stripe of brilliant orange-red color as do the other Xanda leaves, typically being colored orange-red RHS 34A.

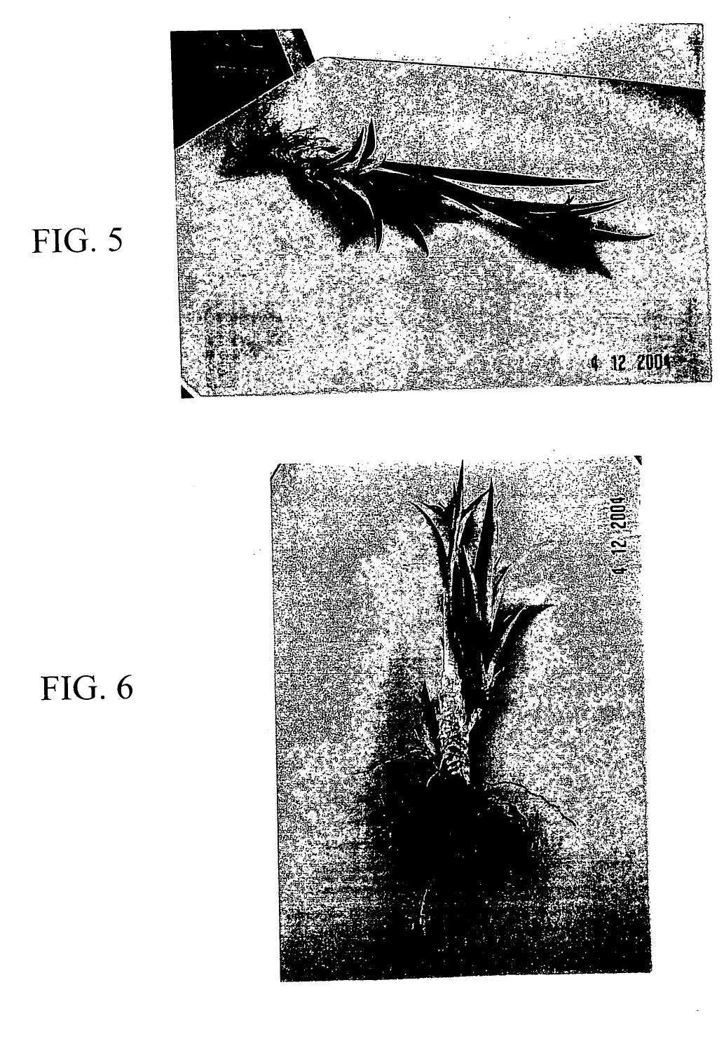

FIG. 5 is a color photograph of an Ananas Lucidus Miller ‘Xanda” showing the peduncle coloration and roots. The ‘Xanda’ stalk is long and colored green-yellow RHS 1D. The peduncle is that portion of the plant which extends from the stalk to the fruit. The peduncle starts green-yellow RHS 1D, turns orange-red RHS 35B from the middle onwards, and ends red RHS 39A.

FIG. 6 is a color photograph of an Ananas Lucidus Miller ‘Xanda” showing the roots. The specimen depicted in FIG. 6 is ten months old.

The following Botanical Description describes plants grown in Sao Pedro, Paracuru-Ceara, Brazil. The plant has not been observed under all possible environmental conditions. The phenotype may vary somewhat with variations in environment such as temperature, light intensity and/or fertilizer rate, without, however, any variance in genotype.

The Ananas Lucidus Miller ‘Xanda’ plant comprises the following parts:

1. Stalk or main shaft, where the rest of the plants' organs are attached to or rest upon.

2. Root System, fascicled with eventual roots, divided in two categories: axillary (found in the axil of the basal leaves) and the underground (originated in the inferior part of the stalk, below the ground).

3. Lance-shaped leaves and straight pipes (Canaliformes) wrapped around the stalk, thornless, with the exception of the terminal thorn colored grey-orange RHS 166C, the outer skin of the superior leaves presented a uniform waxy layer, smooth and has a reddish color in the central part of the leaf and dark reddish at the edges. The inferior outer skin is composed of grooves in the longitude direction, where there can be found the stomas. It is covered by tricomas peltadosescalformes, composed by a white-silverish and continuous fuzz.

Leaf upper surfaces are brilliant red-orange in the middle, ranging from red RHS 48B at the base through red RHS 43A at mid leaf to red RHS 46A close to the tip, with dark brown RHS 200A borders, lightening towards the tip. Leaf lower surfaces are greyed-green RHS 197A, with dull red stripes RHS 48C at the base, RHS 48A at midleaf, and 52B toward the tip.

4. Peduncle: The part of the plant that develops like an extension of the stalk making a connection between the stalk and the fruit, sustaining the inflorescence and later on the green colored fruit at the base and the red in the middle and the top, with the presence of tricomas, presenting a great quantity of offspring. The peduncle peduncle starts green-yellow RHS 1D, turns orange-red RHS 35B from the middle onwards, and ends red RHS 39A.

5. Sapling or seedlings that produce axillary buds and classified according with the position of the plant, in seedling (found in the connection if the peduncle with the base of the fruit), sapling-seedling (found in the connection of the peduncle with the base of the stalk), and sapling (found in the inferior part of the shaft, being airborne if it is above the ground and underground if below the ground).

6. The inflorescence originates from the apical meristem of the stalk, being a spike type, where the concresent flowers are sessile (deprived of the peduncle) and are inserted along a shaft exposed in a spiral, the flowers are hermaphrodites and are located in the axial of a floral bract, dense, pulpous and has a green coloring at the base, with a red coloring at the edges. They are trimerous, with three petals, white at the base and purple at the apex, including three pulpous sepals with a green color at the base, and a red coloring at the edges, with six stamens, exposed in two floral verticals of three, and has a trilocular ovary pistil, and as style and three stigmas.

7. The fruit is known as a fructescene, a composed fruit, a sorosis type, formed by the coalescene of the individual fruits (berries), berrylike fruits, inserted in a central shaft being-exposed in a spiral, composed by a carpology unit, with a cylindrical form, it has a low sappiness, weak aroma, the shin of the fruit originated from the union of the bracts and the sepals. The fruit is red RHS 48C-49A.

8. The crown is a continuation of the growth by the fruit producing new leaves giving as a product an erect crown elevating the fruit. The plant is moderately susceptive to fusariose, doesn't tolerate soaking, is susceptive to wither because of the pela cochinilha.

Growth Cycle: The Ananas Lucidus Miller ‘Xanda’ grows erectly, herbaceous and semi-perennial, about 70-110 cm long. The roots appear at the stem. The roots at the lower part of the stem penetrate the ground and the roots at the higher part surround the stem. The secondary roots develop themselves at the pericycle of the main root (adventical roots), after emerging from the stem. The stem develops itself from the semi-ellipsoidal pulp of the young plant used in the planting. As new leaves develop themselves in their apical meristem, the stem grows longer and thicker. The leaves originate as lateral projections from the apical region of growth from the shaft of the stem, and develop in the direction of the apex. The inflorescence appears from the difference of the apical meristem. With the appearance of the inflorescence the rosette leaves gradually become distinguishable from the peduncle. Each eye represents an extreme of each individual flower, and develops into a berry-like fruit.

All color codes refer to “The Royal Horticultural Society London, 2001 Ed.”. Colors and leaf size may vary somewhat depending on horticultural practices such as light levels and fertilizer rates, among other things, without however, any variance in genotype.

I claim:

1. A new and distinct cultivar of Ananas Lucidus Miller plant named ‘Xanda’, as herein described and illustrated.
